(19) United States
(12) Patent Application Publication
(10) Pub. No.: US 2009/0054523 A1
(43) Pub. Date: Feb. 26, 2009

(54) Use of a Fatty Acid Composition Containing DHA for the Production of a Medical Product or a Food Stuff for the Treatment of Amyloidos-Related Diseases

(76) Inventors: Morten Bryhn, Svelvilk (NO); Knut Sletten, Oslo (NO); Gunilla Torstensdotter Westermark, Rimforsa (SE); Per Olof Westermark, Rimforsa (SE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/795,800

(22) PCT Filed: Jan. 24, 2006
(86) PCT No.: PCT/IB06/00106

§ 371 Date: May 13, 2008

Related U.S. Application Data
(60) Provisional application No. 60/645,604, filed on Jan. 24, 2005.

(30) Foreign Application Priority Data
Jan. 24, 2005 (SE) 0500174-8

(51) Int. Cl. A61K 31/202; A61P 25/00

(52) U.S. Cl. 514/560

(57) ABSTRACT

A method for the treatment and/or prevention of amyloidos-related diseases, such as for example Alzheimer's disease and IgA nephropathy, comprising administering to a human or an animal a composition comprising at least (all-Z omega-3)-4,7,10,13,16,19-docosahexaenoic acid (DHA) is provided.

### TECHNICAL FIELD OF THE INVENTION

The present invention comprises a number of aspects. According to the first aspect of the present invention, a use of a new medicinal product for the treatment and/or prevention of amyloidos-related diseases, is disclosed. According to a second aspect of the present invention, a use of a food stuff or food supplement for the treatment and/or prevention of amyloidos-related diseases, is disclosed. Moreover, according to a third aspect of the invention, a method of for the treatment and/or prevention of amyloidos-related diseases, is disclosed. Finally, according to a fourth aspect of the present invention, a method for treatment and/or prevention of Alzheimer's disease is disclosed. The aspects above are based on at least one of the following features: a fatty acid composition comprising at least (all-Z omega-3)-4,7,10,13,16,19-docosahexaenoic acid (DHA), or a fatty acid composition comprising a combination of (all-Z omega-3)-5,8,11,14,17-eicosapentaenoic acid (EPA), and (all-Z omega-3)-4,7,10,13,16,19-docosahexaenoic acid (DHA).

### BACKGROUND ART

The biological function of a protein depends on its three-dimensional structure, which is determined by its amino acid sequence during the process of protein folding. Normal folding is needed for successful cell functioning and therefore important in maintaining health. Several types of diseases have been found where protein misfolding and conformational change are the main causes of appearance and progression of the diseases (1).

Misfolding of proteins may lead to formation of so called fibrils. Proteins or fragment of proteins are converted from their normally soluble forms to insoluble fibrils or plaques, which accumulate in a variety of organs. The final forms of these aggregates often have a well-defined pathological anatomical appearance, known as amyloid.

Despite the range of proteins involved with their unique and characteristic native folds, the fibrils of the amyloid in which they are found in the disease states are extremely similar in their overall appearance. Proteins known to have the propensity of fibrillar conformation in humans, called precursor proteins, are making up a list of 21 exponents (3) and the number is increasing. Usually the protein in the fibril is made up of a small number of amino acids on average around 20-60 grouping them in the category of polypeptides rather than proteins.

Proteins are usually made up of an alfa-helix and a beta-sheet. Amyloid fibrils, however, usually contain beta-sheet material only rendering the molecules physical properties different from the parent protein. While normal proteins are subjected to a continuous process of degradation by proteolysis, one very important feature of fibrils is the ability, once formed, to be essentially indestructible under physiological conditions. The amyloid fibrils are dominated by hydrogen bonding between the amid and the carbonyl groups of the main chain, rather than by specific interactions of the side chains, which determine the structure of normal proteins. This abnormal bonding induced by the large number of hydrogen bonds of the beta-sheet that must be disrupted to rescue the polypeptide chain from the aggregated state, results in a high resistance to degradation and properly removal from the tissue of deposition.

While in alfa-helices the hydrogen bonds are between side groups within the same strand, in beta sheets the bonds are between one strand and another. Since the second beta-strand can come from a different region of the same protein or from a different molecule, formation of beta-sheets is usually stabilised by protein oligomerisation or aggregation. In this manner the misfolded protein self-associates and become deposited in amyloid aggregates in diverse organs, inducing tissue damage and organ dysfunction. An important part of the deposition process is that a critical concentration of the precursor proteins has to be present before fibril formation occurs (4). It also seems that as soon as an amyloid nucleus has been created the process of aggregation and deposition of amyloid material escalates.

Many of the precursor proteins are not directly prone to fibril deformation. However, when peptide fragments of the precursor protein dissociate from the parent molecule such peptides do not have a stable globular fold to protect them against aggregation. Folding of proteins is a function of physical properties inherent from the amino acid sequence of the chain. These so called non-covalent interactions are weak bonding forces, however, the large number of individual contacts within a protein adds up to a large energy factor favouring normal protein folding. The most important force is the hydrophobic interaction but even hydrogen bonds mentioned above are extremely important. Examples of even weaker forces are electrostatic interactions and van der Waals forces. The number of non-covalent interactions is to some degree a function of the protein chain length meaning that splicing of a section of the protein to a peptide will render the peptide with less stability due to the lower number of non-covalent interactions. The normal folding forces will be weaker which could favour the formation of fibrils.

Less known but significantly important for normal folding as well as maintenance of a stable three-dimensional structure, is protein acylation by covalent attachment of fatty acids (5). It is well established that the protein albumin is able to bind several molecules of fatty acids. Saturated fatty acids such as stearic, palmitic and myristic acid are the predominant fatty acids that attach to proteins in eukaryotic cells (6). From studies using radiolabelled fatty acids we know that each fatty acid labels a different sub-population of proteins with the fatty acid interacting with basic amino acids such as lysine, glycine and arginin. The carboxyl group of the fatty acid forms a salt bridge or a hydrogen bond with basic amino acid side chains. All sites have cylindrical hydrophobic channels of varying shape that force the saturated fatty acids to assume a nearly linear configuration. However, the binding pockets are large enough to accommodate unsaturated fatty acids such as oleic acid and arachidonic acid (7).

Interestingly established amyloid also contains a certain amount of fatty acids. By methanol extraction of amyloid derived from transthyretin about 10% of the dry mass was soluble pointing to the presence of a lipid fraction (8). Gas-chromatography revealed the presence of mixtures of saturated fatty acids like those mentioned above, but also to polyunsaturated fatty acids like palmitoleic acid, linoleic acid, alfa-linolenic acid and arachidonic acid. This pattern of fatty acids is typical for a modern Western diet, which is very much based on saturated fat from dairy products and meat together with seed derived oils. It is quite clear that fatty acids have a function in the normal folding of proteins. The reason why fatty acids are found in amyloid is obscure but interestingly enouth the fatty acids found are congruent with the fats of our diet. One hypothesis is based on the assumption that some fatty acids bound to the polypeptide or protein have weaker affinity rendering the chain less stable and therefore prone to fibrillar deformation.

Amyloid deposits can be reabsorbed and organ function reversed if the synthesis of amyloidogenic protein is shut down. There seems to be a fine balance between the rate at which amyloid is formed and its clearance. It may therefore be possible to promote the resorption of amyloid by reducing the concentration of the amyloidogenic protein to a level below a critical threshold without necessarily eliminating the precursor (AA). Studies of the mechanism of conversion from normally soluble precursor proteins into amyloid fibrils have benefited from the fact that the transition can be reproduced under laboratory conditions. In vitro experiments have demonstrated that conversion of native, fully folded protein into a highly amyloidogenic, partially folded conformer could be blocked by stabilizing native proteins with a specific ligand (9). Other experiments using native precursor proteins such as tau-protein (10) and islet amyloid polypeptide (IAPP) (11) have shown a stimulating effect of certain fatty acids on the assembly of fibrils and amyloid. All long-chain fatty acids tested enhanced assembly to some extent, although greater stimulation was associated with unsaturated forms. Both articles concluded that polyunsaturated fatty acids such as arachidonic acid, oleic acid and linoleic acid but also myristic acid exerted pronounced effects on fibril and amyloid formation. It seemed therefore that common unsaturated fatty acids in our diet could stimulate the formation of fibrils and amyloid and consequently increase the risk of inducing disabling diseases like Alzheimer's dementia, diabetes type 2 and renal failure.

### SUMMARY OF THE INVENTION

Based on the present invention a number of aspects are presented in the appended claims. These aspects are;
1. Use of a new medical product for the treatment and/or prevention of amyloidos-related diseases, such as Alzheimer's disease, IgA nephropathy and type II diabetes.
2. Use of a food stuff or food supplement for the treatment and/or prevention of amyloidos-related diseases.
3. A method for treatment and/or prevention of amyloidos-related diseases.
4. A method for specific treatment of Alzheimer's disease, preferably due to prevention against fibril formation and/or reduction of deposed fibrils or plaques, known as amyloid.

The aspects above are based on at lest one of the following features:
a fatty acid composition comprising at least (all-Z omega-3)-4,7,10,13,16,19-docosahexaenoic acid (DHA).
a fatty acid composition comprising at least a combination of (all-Z omega-3)-4,7,10,13,16,19-docosahexaenoic acid (DHA), and (all-Z omega-3)-5,8,11,14,17-eicosapentaenoic acid (EPA).

According to a first aspect of the invention, the invention relates to the use of a fatty acid composition comprising at least (all-Z omega-3)-4,7,10,13,16,19-docosahexaenoic acid (DHA) for the production of a medicinal product for the treatment and/or prevention of amyloidos-related diseases. From research leading to the invention it was surprisingly found that a fatty acid composition according to the invention prevents formation of so called fibrils or plaques, and/or reduces deposed fibrils or plaques, known as amyloid. Moreover, a fatty acid composition according to the invention predominantly containing DHA seemed to prevent and/or delay formation of fibrils most effectively, wherein DHA may act as an antagonist. At the same time, a fatty acid composition containing at least a combination of the two fatty acids DHA and EPA also demonstrates preventive effect against fibril formation.

Moreover, the treatment according to the invention could be preventive reducing the propensity of fibril formation as well as therapeutic in situations with established amyloid.

Further, under unfavourable conditions, proteins or fragment of proteins are converted from their normally soluble forms to insoluble fibrils or plaques, which accumulate in a variety of organs including the liver, kidneys, spleen, brain, and internal secretory glands like the beta cells of the pancreas inducing toxic effects on cells and tissue. The final forms of these aggregates often have a well-defined pathological anatomical appearance, known as amyloid. This is the reason for the use of the term amyloidoses to describe many of the clinical conditions with which deposition of amyloid are associated. Thus, as used herein, the term “amyloidos-related” diseases means clinical conditions or diseases with which deposition of amyloid, preferably as a consequence of fibril formation, are associated, such as for instance Alzheimer's Dementia, type II diabetes, IgA nephropathy, kidney amyloidoses secondary to chronic inflammatory diseases and Parkinson's disease.

In a preferred embodiment, the fatty acid composition according to the invention further comprises (all-Z omega-3)-5,8,11,14,17-eicosapentaenoic acid (EPA). This means, a fatty acid composition comprising at least a combination of DHA and EPA for the treatment and/or prevention of amyloidos-related diseases. In a more preferred embodiment of the invention, the invention relates to the use of a fatty acid composition, wherein the weight ratio of EPA:DHA in the fatty acid composition is 1:X, where X is equal or greater than 1. Please note that X being one of an integer or non-integer.

Moreover, at mentioned before, a preferred effect of the invention is accomplished by a fatty acid composition rich in DHA. The term “rich” herein includes more or less a fatty acid composition primary containing DHA (none EPA) and a fatty acid composition where the amount of DHA≧EPA. Further, the term “amount” herein relates to weight or volume of the fatty acid composition.

Moreover, the desired pharmacological and/or therapeutic effect may be achieved by the fatty acid composition according to the invention.

In a preferred embodiment of the invention, EPA and DHA in the fatty acid composition are present in the composition in an EPA:DHA ratio from 1:1 to 1:8. In a more preferred embodiment the EPA:DHA ratio in the fatty acid composition is from about 1:1 to 1:6. In a further embodiment of the invention, the fatty acid composition is a DHA-product.

Moreover, in another embodiment, the fatty acids in the composition according to the invention is presented in at least one of esterified form, ethyl ester form, salt form and free acid form, or any combinations thereof. In a preferred embodiment, the fatty acid composition is comprised of a combination of EPA and DHA in triglyceride form.

In another embodiment, at least DHA is obtained from at least one of vegetable, microbial and animal origins, or combinations thereof. Moreover, in a further embodiment, wherein the fatty acid composition comprising at least a combination of DHA and EPA, at least one of DHA and EPA is obtained from at least one of vegetable, microbial and animal origins or combinations thereof. The medicinal product or pharmaceutical product includes therefore for instance a fatty acid composition comprising at least one of a DHA-containing microbial oil and a mixture of an DHA-containing oil from microbial origin and a EPA-containing oil from a marine origin. Moreover, the fatty acid composition according to the invention may additionally also comprise at least one of arachidonic acid (ARA), docosapentaenoic acid, heneicosapentaenoic acid and octadecatentraenoic, or any combinations thereof. Suitably, at least a part of the EPA and/or DHA is produced from a marine oil, preferably a fish oil. Furthermore, in another embodiment of the medicinal product, the fatty acid composition is produced from a marine oil, such as a fish oil.

In a specific embodiment of the invention, the fatty acid composition is comprised of at least a combination of EPA and DHA in triglyceride form. Moreover, it should be pointed out that the fatty acid composition is administered to a human or an animal, preferably orally. However, the medicinal product according to the invention may also be produced for administration though any other route where the active ingredients may be efficiently absorbed and utilized, e.g. intravenously, subcutaneously, intramuscularly, intranasally, rectally, vaginally or topically.

In another embodiment, said fatty acid composition is administered in an amount providing a daily dosage of 1 g to 15 g of said fatty acid composition for a human. In a preferred embodiment between 2 and 10 g of said fatty acid composition is administered per day, and in a more preferred embodiment between 2 and 8 g of said fatty acid composition. The medicinal product or pharmaceutical composition or pharmaceutical preparation according to the invention may also comprise other substances such as an inert vehicle, or pharmaceutical acceptable adjuvants, carriers, preservatives etc., which are well known in to those skilled in the art. However, the medicinal product may also be administered to an animal, such as a pet or a horse. Moreover, it should be pointed out that the medicinal product may be at least one of an amyloid-preventing agent or amyloid-deposit decreasing agent.

In another embodiment of the invention, the invention relates to a use of a fatty acid composition comprising at least (all-Z omega-3)-4,7,10,13,16,19-docosahexaenoic acid (DHA) that upon administration to a human or an animal will prevent the formation of aggregates of protein fibrils or plaque and/or reduce deposed fibrils, for the production of a medicinal product for the treatment and/or prevention of amyloidos-related diseases.

Furthermore, in an embodiment of the invention, said amyloidos-related disease is Alzheimer's dementia.

In another embodiment of the invention, said amyloidos-related disease is IgA nephropathy. In a specific embodiment of the invention, a fatty acid composition comprising a combination of at least DHA and EPA is used for the treatment and/or prevention of IgA nephropathy. Moreover, the invention also includes use of a fatty acid composition comprising an effective amount of at least DHA or a combination of EPA or DHA that upon administration to a human or an animal preferable will prevent the formation of aggregates of protein fibrils or plaque and/or reduce deposed fibrils, for the production of a medicinal product for the treatment and/or prevention of IgA nephropathy.

In another embodiment of the invention, the invention relates to the use of a fatty acid composition comprising at least (all-Z omega-3)-4,7,10,13,16,19-docosahexaenoic acid (DHA) for the production of a medicinal product for the treatment and/or prevention of amyloidos-related diseases, wherein the disease is type II diabetes mellitus. In a preferred embodiment, said fatty acid composition further comprises (all-Z omega-3)-5,8,11,14,17-eicosapentaenoic acid (EPA), i.e. the fatty acid composition comprising at least a combination of EPA and DHA for treatment of type II diabetes. Moreover, in a specific embodiment, the amount of DHA is≧the amount of EPA.

Furthermore, in another embodiment of the invention the amyloidos-related diseases is at least one of amyloidoses, Parkinson's disease, amyotrophic lateral sclerosis, the spongiform encephalopathies such as Creutzfeld-Jacob disease, cystic fibrosis, kidney amyloidoses secondary to inflammatory diseases and renal amyloidoses, and amyloid deposition in myocardium and neural tissue.

According to a second aspect of the invention, the present invention relates to the use of a fatty acid composition comprising at least (all-Z omega-3)-4,7,10,13,16,19-docosahexaenoic acid (DHA) for the production of a food stuff or food supplement for the treatment and/or prevention of amyloidos-related diseases. From research leading to the invention it was surprisingly found that a fatty acid composition according to the invention prevents formation of so called fibrils or plaques, and/or reduces deposed fibrils or plaques, known as amyloid. The most preferred effect of the invention is also here accomplished by the use of at least DHA or a fatty acid composition rich in DHA. DHA seemed to prevent and/or delay formation of fibrils most effectively. At the same time, a fatty acid composition containing at least a combination of the two fatty acids DHA and EPA also demonstrates preventive effect against fibril formation. Moreover, the use according to above leads to the same advantages and possibilities as mentioned before. The treatment according to the invention could be preventive, reducing the propensity of fibril formation, as well as therapeutic in situations with established amyloid. The definition concerning “amyloidos-related” diseases is also included for the patent positions related to a food stuff or a food supplement according to the invention.

One advantage of manufacturing and selling a food stuff for the treatment and/or prevention of amyloidos-related diseases is that such a food stuff will be more easily accessible for people. In preventive purpose they preferably buy the product or supplement in a health store and/or a supermarket, and they do not need to visit a doctor.

In a specific embodiment, the present invention relates to the use of a fatty acid composition comprising at least (all-Z omega-3)-4,7,10,13,16,19-docosahexaenoic acid (DHA) and (all-Z omega-3)-5,8,11,14,17-eicosapentaenoic acid (EPA) for the production of a food stuff or food supplement for the treatment and/or prevention of amyloidos-related diseases.

In a more preferred embodiment of the invention, the invention relates to the use of a fatty acid composition, wherein the weight ratio of EPA:DHA in the fatty acid composition is 1:X, where X is equal or greater than 1. Please note that X being one of an integer or non-integer. Moreover, a preferred effect of the invention concerning weight reduction is accomplished by a fatty acid composition rich in DHA. The term “rich” herein includes more or less a fatty acid composition primary containing DHA (none EPA) and a fatty acid composition where the amount of DHA≧EPA. Further, the term “amount” herein relates to weight or volume of the fatty acid composition.

Moreover, the desired pharmacological and/or therapeutic effect may be achieved by the fatty acid composition according to the invention.

In another embodiment of the invention, EPA and DHA in the fatty acid composition are present in the composition of an EPA:DHA ratio from 1:1 to 1:8. In a more preferred embodiment the EPA:DHA ratio in the fatty acid composition is from about 1:1 to 1:6. In a specific embodiment of the invention, the fatty acid composition is a DHA-product.

Moreover, in another embodiment, the fatty acids in the composition according to the invention is presented in at least one of esterified form, ethyl ester form, salt form and free acid form, or any combinations thereof. In a preferred embodiment, the fatty acid composition is comprised of a combination of EPA and DHA in triglyceride form.

Further, in another embodiment, at least DHA is obtained from at least one of vegetable, microbial and animal origins, or combinations thereof. In preferred embodiment, wherein the fatty acid composition comprising at least a combination of DHA and EPA, at least one of DHA and EPA is obtained from at least one of vegetable, microbial and animal origins or combinations thereof. The food stuff or food supplement includes therefore, for instance, a fatty acid composition comprising at least one of a DHA-containing microbial oil and a mixture of an DHA-containing oil from microbial origin and a EPA-containing oil from a marine origin. Further, the fatty acid composition according to the invention may additionally also comprise at least one of arachidonic acid (ARA), docosapentaenoic acid, heneicosapentaenoic acid and octadecatentraenoic or derivatives thereof, or any combinations thereof. Suitably, at least a part of the EPA and/or DHA is produced from a marine oil, preferably a fish oil. Furthermore, in another embodiment of the food stuff or food supplement, the fatty acid composition is produced from a marine oil, such as a fish oil.

In a preferred embodiment of the invention, the fatty acid composition is comprised of at least a combination of EPA and DHA in triglyceride form. Moreover, it should be pointed out that the fatty acid composition is administered to a human or an animal, preferably orally. However, the food stuff or food supplement according to the invention may also be produced for administration though any other route as mentioned before.

In a specific embodiment of the invention, the food stuff or food supplement is in form of a capsule.

Preferably, the capsule is flavoured. More preferably, the capsule is a gelatine capsule which is flavoured. This embodiment also includes a capsule, therein both the capsules and the encapsulated fatty acid composition are flavoured. By flavouring the capsule as mentioned above, the capsule becomes more attractive to the user.

In another preferred embodiment, said fatty acid composition is administered in an amount providing a daily dosage of 1 g to 15 g of said fatty acid composition for a human. In a more preferred embodiment between 2 and 10 g of said fatty acid composition is administered per day, and in a most preferred embodiment between 2 and 8 g of said fatty acid composition. The food stuff or food supplement according to the invention may also comprise other substances such as an inert vehicle, or pharmaceutical acceptable adjuvants, carriers, preservatives etc., which are well known in to those skilled in the art. Additionally, even the food stuff or food supplement may be administered to an animal such as a pet or a horse. Moreover, it should be pointed out that the food stuff or food supplement may be at least one of an amyloid-preventing agent or amyloid-deposit decreasing agent.

In a specific embodiment, the present invention relates to use of a fatty acid composition comprising at least (all-Z omega-3)-4,7,10,13,16,19-docosahexaenoic acid (DHA), that upon administration to a human or an animal will prevent the formation of aggregates of protein fibrils or plaque and/or reduce deposed fibrils, for the production of a medicinal product for the treatment and/or prevention of amyloidos-related diseases. Furthermore, in another embodiment, said amyloidos-related disease is Alzheimer's dementia or Alzheimer's disease. The term Alzheimer's also includes persons who are at risk of, or exhibits the symptoms of Alzheimer's disease.

Moreover, one advantage of selling a food stuff for prevention of preferably Alzheimer's disease or Parkinson's disease is that the food stuff may help people not to develop these diseases in the future.

In addition, in another embodiment of the invention, said amyloidos-related disease is IgA nephropathy. In a preferred embodiment of the invention, a fatty acid composition comprising a combination of at least DHA and EPA is used for the treatment and/or prevention of IgA nephropathy. Moreover, the invention also includes use of a fatty acid composition comprising an effective amount of at least DHA or a combination of EPA or DHA, that upon administration to a human or an animal preferable will prevent the formation of aggregates of protein fibrils or plaque and/or reduce deposed fibrils, for the production of a food stuff or food supplement for the treatment and/or prevention of IgA nephropathy.

In an other embodiment of the invention, the invention relates to the use of a fatty acid composition comprising at least (all-Z omega-3)-4,7,10,13,16,19-docosahexaenoic acid (DHA) for the production of a food stuff or food supplement for the treatment and/or prevention of amyloidos-related diseases, wherein the disease is type II diabetes mellitus. In a referred embodiment, said fatty acid composition further comprises (all-Z omega-3)-5,8,11,14,17-eicosapentaenoic acid (EPA), i.e. the fatty acid composition comprising at least a combination of EPA and DHA, for treatment of type II diabetes. Moreover, in a specific embodiment, the amount of DHA≧EPA.

Furthermore, in another embodiment of the invention the amyloidos-related diseases is at least one of amyloidoses, Parkinson's disease, amyotrophic lateral sclerosis, the spongiform encephalopathies such as Creutzfeld-Jacob disease, cystic fibrosis, kidney amyloidoses secondary to inflammatory diseases, renal amyloidoses, and amyloid deposition in myocardium and neural tissue.

According to a third aspect of the invention, the present invention relates to a method for the treatment and/or prevention of amyloidos-related diseases, wherein an effective amount of a fatty acid composition comprising at least (all-Z omega-3)-4,7,10,13,16,19-docosahexaenoic acid (DHA) is administered to a human or an animal. Herein, “an effective amount” also includes a therapeutically or a pharmaceutically active amount of the fatty acid composition. This expression relates to a dose of said fatty acid composition that will lead to the desired pharmacological and/or therapeutic effect. The desired pharmacological and/or therapeutic effect is, as stated above, achieved by the fatty acid composition according to the invention. From research leading to the invention it was surprisingly found that a fatty acid composition according to the invention prevents formation of so called fibrils or plaques, and/or reduces deposed fibrils or plaques, known as amyloid.

In a preferred embodiment of the invention, the fatty acid composition further comprises (all-Z omega-3)-5,8,11,14,17-eicosapentaenoic acid (EPA). In a more preferred embodiment, the weight ratio of EPA:DHA in the fatty acid composition is 1:X, where X is equal or greater than 1. The most preferred effect of the invention is also here accomplished by the use of at least DHA or a fatty acid composition rich in DHA. At the same time, a fatty acid composition containing at least a combination of the two fatty acids DHA and EPA also demonstrates preventive effect against fibril formation. Moreover, the method leads to the same advantages and possibilities as mentioned before. Thus, the embodiments described above are also included for the method according to the invention concerning treatment and/or prevention of amyloidos-related diseases, such as Alzheimer's disease, IgA nephropathy, type II diabetes mellitus, amyloidoses, Parkinson's disease, kidney amyloidoses secondary to chronic inflammatory disease and Creutzfeld-Jacob disease.

According to a fourth aspect of the invention, the invention relates to a method for treatment and/or prevention of Alzheimer's disease, wherein a fatty acid composition comprising at least a combination of (all-Z omega-3)-5,8,11,14,17-eicosapentaenoic acid (EPA) and (all-Z omega-3)-4,7,10,13,16,19-docosahexaenoic acid (DHA) is administered to a human or an animal. Preferably, the administration to a patient prevents formation of aggregates of protein fibrils, or misfolding of proteins or fragments, and/or decreases deposits of amyloid.

In a preferred method for treatment of Alzheimer's disease according to the invention, the disease is caused by deposition of amyloid. In a specific embodiment of the invention, the method of treating Alzheimer's disease is related to administering to a human suffering from the disease an effective Alzheimer's disease alleviating amount of a amyloid-deposit-decreasing agent. Preferably, the amyloid-deposit-decreasing agent is a fatty acid composition comprising at least one of a combination of the fatty acids DHA and EPA.

In another embodiment of the invention, the embodiment relates to use of a fatty acid composition comprising at least a combination of DHA and EPA, that upon administration to a human or an animal will lead to prevention of, or reduction of, deposition of amyloid, for the manufacture of a medicament or a food supplement, for the treatment and/or prevention of Alzheimer's disease, preferably caused by amyloidosis. In a specific embodiment of the invention, the fatty acid composition comprising at least 70% unsaturated omega-3 fatty acids wherein the fatty acids DHA and EPA are present in a weight ratio from about 1:2 to 2:1. Moreover, in a more preferred embodiment of the invention, the invention relates to the use of a fatty acid composition, wherein the weight ratio of EPA:DHA in the fatty acid composition is 1:X, where X is equal or greater than 1. Please note that X being one of an integer or non-integer. Moreover, a preferred effect of the invention is accomplished by a fatty acid composition rich in DHA. The term “rich” herein includes more or less a fatty acid composition primary containing DHA (none EPA), and a fatty acid composition where the amount of DHA≧EPA. Further, the term “amount” herein relates to weight or volume of the fatty acid composition. Moreover, the desired pharmacological and/or therapeutic effect may be achieved by the fatty acid composition according to the invention.

In another embodiment, the fatty acids in the composition, according to the invention, are presented in at least one of esterified form, ethyl ester form, salt form and free acid form, or any combinations thereof. In a preferred embodiment, the fatty acid composition is comprised of a combination of EPA and DHA in triglyceride form. Moreover, in a further embodiment, wherein at least one of DHA and EPA, is obtained from at least one of vegetable, microbial and animal origins or combinations thereof. The medicinal product or pharmaceutical product includes therefore, for instance, a fatty acid composition comprising at least one of a DHA-containing microbial oil and a mixture of an DHA-containing oil from microbial origin and a EPA-containing oil from a marine origin. Furthermore, the fatty acid composition according to the invention may additionally also comprise at least one of arachidonic acid (ARA), docosapentaenoic acid, heneicosapentaenoic acid and octadecatentraenoic, or any combinations thereof. Suitably, at least a part of the EPA and/or DHA is produced from a marine oil, preferably a fish oil. Suitable, the fatty acid composition is produced from a marine oil, such as a fish oil.

In a preferred embodiment of the invention, the fatty acid composition is comprised of at least a combination of EPA and DHA in triglyceride form. Moreover, it should be pointed out that the fatty acid composition is administered to a human or an animal, preferably orally. However, the fatty acid composition according to the invention, may also be produced for administration though any other route where the active ingredients may be efficiently absorbed and utilized, e.g. intravenously, subcutaneously, intramuscularly, intranasally, rectally, vaginally or topically.

In another preferred embodiment, said fatty acid composition is administered in an amount providing a daily dosage of 1 g to 15 g of said fatty acid composition for a human. In a more preferred embodiment between 2 and 10 g of said fatty acid composition is administered per day, and in a most preferred embodiment between 2 and 8 g of said fatty acid composition. Additionally, the preparation according to the invention may also comprise other substances such as an inert vehicle, or pharmaceutical acceptable adjuvants, carriers, preservatives etc., which are well known to those skilled in the art. Moreover, it should be pointed out that the fatty acid mixture or composition may be at least one of an amyloid-preventing agent or amyloid-deposit decreasing agent. In addition, the invention also relates to a method for treatment of Alzheimer's disease due to prevention of misfolding of proteins, or fragments, that may lead to formation of so called fibrils or plaques, and/or due to decreasing of amyloid deposits, wherein a fatty acid composition according to the invention is administered to a human.

Finally, according to the fifth aspect of the invention, the invention relates to a method for prevention and/or for treatment of amyloidoses, wherein a fatty acid composition comprising at least (all-Z omega-3)-4,7,10,13,16,19-docosahexaenoic (DHA) is administered to a human or an animal.

In a preferred embodiment of the invention, the fatty acid composition further comprises (all-Z omega-3)-5,8,11,14,17-eicosapentaenoic acid (EPA). Moreover, in a specific embodiment of the invention the weight ratio of EPA:DHA in the fatty acid composition is 1:X, where X is equal or greater than 1. From the research leading to the invention it was found that the most preferred effect concerning inhibitation, or prevention, of formation of fibril aggregates is accomplished by a fatty acid composition comprising at least DHA. Furthermore, a fatty acid composition comprising at least DHA and EPA, wherein the amount of DHA≧EPA, also show preventive effect against fibril formation. Thus, this method leads to the same advantages as mentioned before. Moreover the embodiments described before are also include for the method of treating amyloidoses. Finally, the invention also includes use of a fatty acid composition comprising at least DHA preferable at least a combination of DHA and EPA, for prevention of fibrils, plaque, or amyloid aggregates.

As used herein amyloidos-related conditions or diseases, at least includes Alzheimer's disease or dementia, Parkinson's disease, amyotrophic lateral sclerosis, the spongiform encephalopathies such as Creutzfeld-Jacob disease, cystic fibrosis, type II diabetes, renal amyloidoses, IgA nephropaty, and amyloid deposition in myocardium and neural tissue. These diseases can be sporadic, inherited or even infectious, and are often occur only late in life even if inherited forms may appear much earlier. Each disease is associated with a particular protein and aggregates of these proteins are thought to be the direct origin of the pathological conditions associated with the disease. Moreover, the treatment and/or prevention according to administering the fatty acid composition of the invention may also include at least one of; treatment due to reduction of amyloid aggregates, prevention of misfolding of proteins that may lead to formation of so called fibrils or plaque, treatment due to decreasing of the production of Aβ-protein (amyloid beta protein), and prevention and/or treatment due to inhibiting or slow down the formation of protein fibrils, aggregates, or plaque. Moreover, the present invention also includes prevention of fibril accumulation, or formation, by administering a fatty acid composition according to the invention.

In addition, as used herein the term “treatment” means both treatment having a curing or alleviating purpose and the treatment of a amyloidos-related disease can be made either acutely or chronically. By chronically treatment is meant a treatment witch continues for weeks or years.

### BRIEF DESCRIPTION OF THE DRAWINGS

In the studies and examples below reference is made to the accompanying drawings, where the figures concern studies performed in vitro as well as in vivo. The studies were preformed in order to demonstrate that a treatment, with a fatty acid composition comprising at least DHA, prevents fibril and amyloid formation. Herein reference is made to the accompanying drawings, on which:

FIG. 1 shows fibril formation of omega-3 preparations during 30 minutes.

FIG. 2 demonstrates fibril formation between the fatty acid preparations with the most prominent fibril inducing effect, soy oil and olive oil, compared to the fibril preventing DHA-concentrate EPAX 2050 (which comprises approximately 20% EPA and 50% DHA). Moreover,

FIG. 3 shows preventive effect against fibril formation up to at least 150 minutes comparing different omega-3 preparations.

FIG. 4 illustrates effects against fibril formation with a DHA-concentrate compared to olive oil and soy oil.

### DESCRIPTION OF PREFERRED EMBODIMENTS

A number of preferred embodiments of the invention were performed in order to demonstrate that a fatty acid composition rich in DHA is effective on the treatment and/or prevention of amyloidos-related diseases.

Fibril formation is a consequence of misfolding the precursor proteins. The reason for this abnormal behaviour of forming normal three-dimensional structures has not been fully elucidated. As discussed in the background art, it could seem that common polyunsaturated fatty acids frequently recommended by dieticians to prevent cardio-vascular disease and cancer, could in fact increase the propensity of misfolding precursor proteins, thereby inducing amyloid deposition. However, the present invention disclose results showing that another marine long-chain polyunsaturated fatty acid, namely docosahexaenoic acid (DHA), surprisingly seemed to indicating a preventive effect on amyloid formation by prolonging the time to spontaneous fibril formation of IAPP.

The present invention discloses the results of experiments with one synthetic precursor protein, synthetic IAPP, spontaneously forming fibrils, as well as semi-vivo experiments with pancreatic islets from transgenic animals producing human IAPP. The invention also discloses results from in vivo experiments in animals where organ amyloid is induced by injections of a pro-inflammatory compound, namely silver nitrate. This peptide was incubated with free fatty acids of marine origin comparing the effects on fibril formation with fatty acids like oleic acid and linoleic acid known to stimulate the formation of amyloid fibrils. Moreover, the present invention also discloses results of experiments in a semi-vivo model, where pancreatic islets from transgenic mice producing human IAPP and spontaneously forming beta-call amyloid were prepared and incubated with oleic acid and docosahexaenoic acid.

It is well established that inflammatory diseases create a high risk of developing amyloid in different organs, a situation called secondary amyloid. A situation of extreme inflammation was created in mice injected weekly with silver nitrate. Different groups of animals were treated with EPAX 1050 (a high DHA omega-3 oil), pure DHA (97% concentrate), or olive oil (containing about 80% oleic acid). A control group was given standard chow.

In a first preferred embodiment, the effects of different omega-3 preparations on fibril formation, were studied.

In a second embodiment, the effects of a fatty acid composition rich in DHA, EPAX 2050 (a high omega-3 oil), an oil comprising at least a combination of DHA and EPA, (K85: approximately 460 mg EPA and 375 mg DHA), and an olive oil, on deposition of amyloid fibrils in pancreatic islets, were studied.

In a third embodiment, the effects of treatment with EPAX 1050, DHA, or oleic acid in animals with secondary amyloid induced by subcutaneous injections of silver nitrate, were studied. Deposition of amyloid was monitored in animals given normal chow. After 25 weeks of silver nitrate injections significant amyloid deposition was established in organs of control animals. The experiment was then terminated and all animals were sacrificed and the spleens were examined for amyloid deposition.

### EXAMPLES

### In Vitro Experiments on Fibril Formation

In the first study, the effects on fibril formation of different omega-3 preparations were studied. The in vitro fibril formation experiments were performed in small glass test tubes. A stock of dissolved fatty acids in ethanol was prepared by dissolving fatty acids in 100% ethanol at a concentration of 10 mM. After mixing with the same amount of concentrated NaOH, the final concentration of ethanol was 3%. The fatty acids or fatty acid combinations in the table below were tested.

| | | 10 mM |
|---|---|---|
| Fatty acids | Mol. weight | contained |
| Linoleic acid | 278 | 27.8 mg |
| (from soybean oil) | | |
| Oleic acid | 280 | 28.0 mg |
| (from olive oil) | | |
| EPAX DHA concentrate, | 301 | 30.1 mg |
| EPAX2050 | | |
| (≈500 mg DHA/g and | | |
| ≈200 mg EPA/g) | | |
| EPAX 4510 | 318 | 31.8 mg |
| (≈450 mg EPA/g) | | |
| EPA 95 | 302 | 30.2 mg |
| (≈950 mg EPA/g) | | |
| K85 | 314 | 31.4 mg |
| (≈465 mg EPA/g and | | |
| ≈375 mg DHA/g) | | |
| Control synthIAPP + ethanol | | |

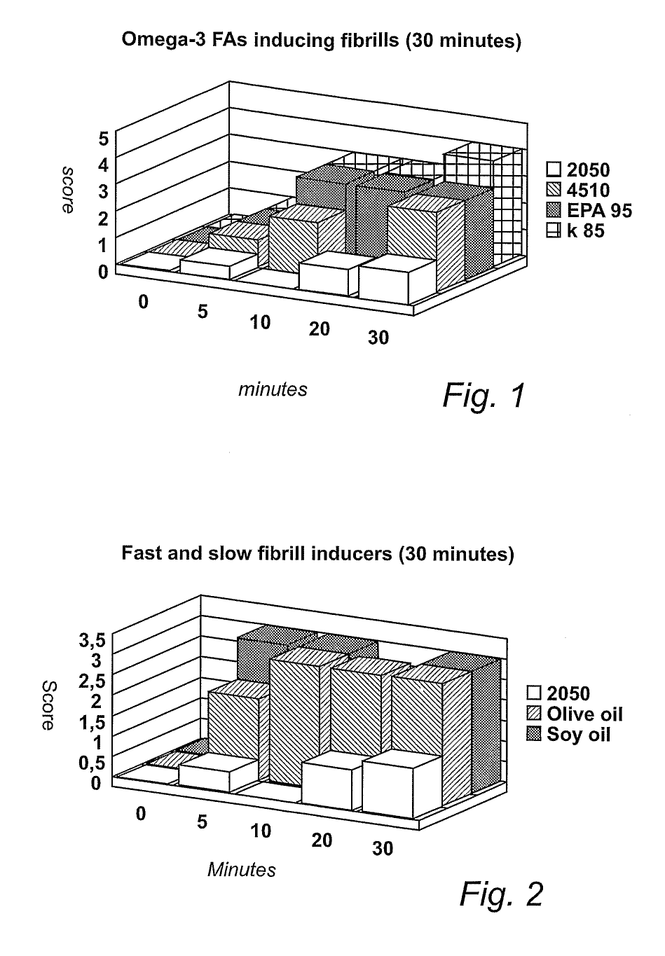

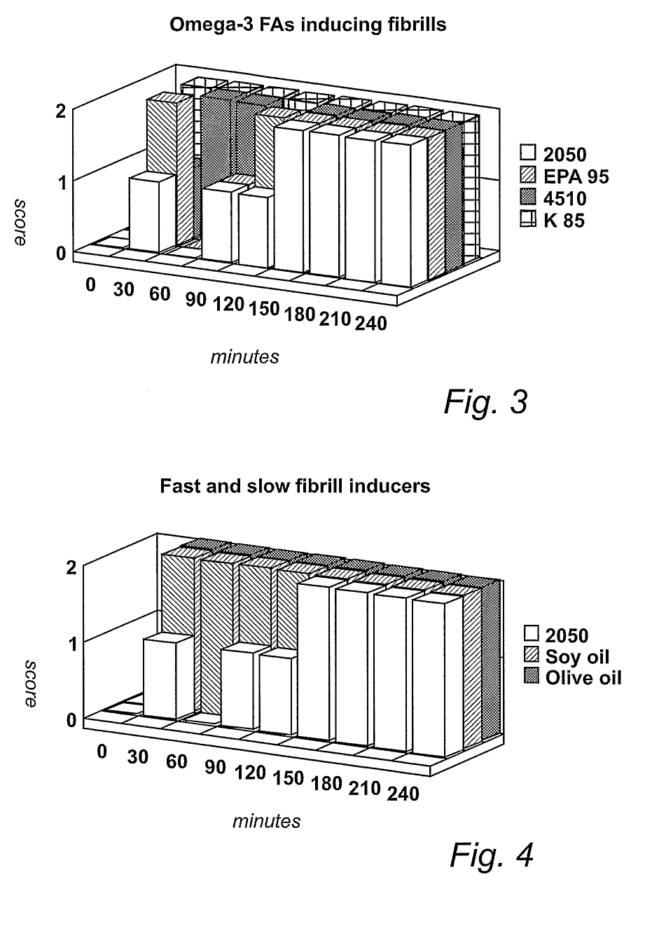

Synthetic precursor protein, synthetic IAPP spontaneously forming filbrils, was dissolved in dimethylsulfoxide (DMSO) at a concentration of 10 mg/ml. 25 μM was incubated with each one of the fatty acids, each one in a concentration of 125 μM, in distilled water. One μl aliquots of each sample were analysed after 5, 10, 20, 30, 60, 90, 120, 150, 180, 210, and 240 minutes in the electron microscope after negative contrasting with 2% uranyl acetate in 50% ethanol. Formation of fibrils was observed by electron microscopical analysis and recorded as scores arbitrarily between 0 and 5 in the 30 minutes experiments and between 1 and 2 in the 240 minutes experiments. FIG. 1 demonstrates fibril formation of the omega-3 preparations during 30 minutes. The omega-3 concentrate containing predominantly DHA, EPAX 2050, seemed to prevent or delay the spontaneously formation of fibrils most effectively. Moreover, FIG. 2 demonstrates fibril formation between the fatty acid preparation with the most prominent fibril inducing effect, olive oil and soy oil, compared to the fibril preventing DHA-concentrate EPAX 2050. In addition, fibril induction was also followed for 240 minutes to quantify the fibril preventive effect of the DHA-concentrate. FIG. 3 demonstrates that preventive effect against fibril formation was evident up till 150 minutes in the experiment comparing different omega-3 preparations. Similar prevention was obtained in the experiment with the DHA concentrate compared to olive oil and soy oil, see FIG. 4.

Thus, this study shows that treatment with a fatty acid composition rich in DHA leads to prevention of fibril or plaque formation. Moreover, it seams that DHA act as an fibril inhibitor. At the same time the invention also shows preventive effect on fibril formation of a product comprising at least a combination of DHA and EPA, wherein preferably the amount of DHA≧EPA. The results also suggest a specific preventive effect against fibril formation of an omega-3 product of marine origin as compared with soy oil and olive oil.

### In Vitro Experiments on Amyloid Formation in Pancreatic Islets

In the second study protective effects on amyloid deposition of an olive oil, a high DHA omega-3 oil, and a composition comprising at least DHA and EPA, wherein the amount of EPA≧DHA, were studied.

Transgenic mice carrying the human IAPP gene may be used for studying deposition of amyloid fibrils in the pancreatic islets (11). Therefore, single pancreatic islets were isolated and cultured from transgenic mice. Pancreas were removed under sterile conditions and placed in Hank's balanced salts and finely minced. Small pieces of tissue were enzymatically digested by collagenase for 10 minutes in a 37 degree Celcius water bath. The islets were individually selected under the microscope. Subsequently the islets were cultured overnight in 24 well cell cluster containing RPMI 1640 medium supplemented with 10% fetal bovine serum, penicillin (100 U/ml), streptomycin (1,1 mg/ml) and 22, 0 mM glucose at 37 degrees in humidified air containing 5% carbonmonoxide.

500 μM of olive oil, high DHA omega-3 oil (EPAX 2050) and K85 oil and 1% fatty acid-free albumin were added to the wells and the islets were cultured in RPMI medium during 2 weeks. There were about 60 islets in each well. Congo red from a stock solution was added to the wells and the islets were examined using light microscopy. Amyloid deposition is stained with Congo red while other cell material does not.

No difference was observed between the different islet groups regarding survival (Table below).

No. of
living islets

| Fatty acid | Start | End |
|---|---|---|
| EPAX 2050 | 60 | 50 |
| K 85 | 60 | 52 |
| Olive oil | 65 | 54 |

The results of this study shows that 4-5% of the cell islets incubated in the olive oil solution were stained with Congo red indicating intracellular amyloid deposition. Surprisingly only 1% of the cells from islets incubated with high DHA, but even also the K85 concentrate, were stained with Congo red indicating a protective effect against amyloid deposition. However, the DHA rich product, EPAX 2050TG, exhibited stronger effect compared to K85 (EPA≧DHA). Additionally, the results of this study confirm effects supporting prevention and treatment of amyloidos-related diseases influenced of a fatty acid composition comprising at least DHA.

### Secondary Amyloidosis in Animals Induced by Silver Nitrate Injections

Secondary amyloidosis can be induced in mice given an inflammatory challenge such as silver nitrate. This was studied in the following.

Female NMRI mice were injected once a week subcutaneously with 0.3 ml of a 1% silver nitrate solution for 25 weeks. The animals were fed high fat diet containing mainly sunflower oil with a fat content of 55% of total daily calories. Three groups each consisting of 6 animals were given different omega-3 fatty acid concentrates (intervention group) or standard olive oil containing about 78% oleic acid (oleic acid group). In the intervention groups 15% of the fat content was exchanged with either 97% docosahexaenoic acid (DHA group) or EPAX1050TG containing 10% of EPA and 50% of DHA (EPAX group).

| DHA group | EPAX group | Oleic acid group |
|---|---|---|
| n = 6 | n = 6 | n = 6 |
| DHA 97% conc. | 10% EPA, 50% DHA | 78% oleic acid |
| 15% of tot. fat | 15% of tot. fat | 15% of tot. fat |

After study completion the mice were sacrificed and spleens were collected. For amyloid demonstration, one half of each spleen was crushed between two glass slides, smeared homogenously over both slides, and air-dried in room temperature overnight, while the other half of the spleen was fixed in 10% buffered neutral formalin solution and embedded in paraffin.

The air-dried smear material and 10-μm-thick sections from the formalin fixed material were stained for amyloid with specific dye alkaline Congo red. The slides were examined in cross-polarized light for bright green birefringence, specific for amyloid. Evaluation of the material was performed on coded slides.

Amyloid appeared in the spleen in three out of five mice fed oleic acid, in one out of six mice fed DHA and in one out of five mice fed EPAX (table below). Two animals died during the study.

| | | Oleic acid |
|---|---|---|
| DHA group | EPAX group | group |
| n = 6 | n = 5 | n = 5 |
| 1/6 animals | 1/5 animals | 3/5 animals |
| with | with | with |
| amyloid | amyloid | amyloid |

It can clearly be seen that secondary amyloidosis developed to a higher degree in mice fed the oleic acid diet when compared to mice fed DHA or EPAX.

However, due to the low number of animals remaining in each study, no conclusive results could be made between the DHA and EPAX group.

### Discussion

In the studies presented above, the synthetic precursor protein IAPP spontaneously forming fibrils were incubated in vitro with a series of fatty acids of the omega-3 series as well as omega-6 and omega-9. The omega-3 concentrate containing predominantly DHA seemed to prevent or delay the spontaneous formation of fibrils while the omega-6 (soybean oil) and omega-9 (olive oil) fatty acids seemed to provoke fibril formation. The last pattern is known from previous experiments (10, 11). The preventive effect of DHA, however, is an unexpected finding. Other omega-3 fatty acids did not have the same effect as DHA even if some preventive effects were observed compared to the omega-6 and 9 oils. Moreover, in the amyloid in vitro model on islets from transgenic IAPP producing mice DHA, but even another high omega-concentrate, K85, induced amyloid deposition to a significant less extent compared to the omega-9 olive oil.

At the same time the invention also shows preventive effect on fibril formation of a product comprising at least a combination of DHA and EPA, wherein preferably the amount of DHA≧EPA.

A preventive effect of amyloid deposition could even be demonstrated in vivo by giving animals with silver nitrate induced secondary amyloidoses different fatty acids or fatty acid combination. After 25 weeks of treatment animals given EPAX1050 or approximately pure DHA did not develop amyloid to the same extent as animals given oleic acid. In fact only one animal in the DHA groups (EPAX1050 and pure DHA) developed amyloid after silver nitrate injections compared to 3 animals in the oleic acid group. Even if the number of animals used is too small for a statistical evaluation, the trend is quite clear meaning that DHA or high DHA concentrates seems to prevent against development of amyloid. The congruence between in vitro derived data and in vivo data is striking indicating a novel therapeutic modality to diseases etc.

The present findings indicate a novel therapeutic modality to diseases caused by amyloid deposition. The treatment could be preventive reducing the propensity of fibril formation as well as therapeutic in situations with established amyloid.

Finally, the results strongly support the use of a medicinal product, a pharmaceutical composition, a food stuff or a food supplement, comprising a fatty acid composition comprising at least DHA, for the treatment and/or prevention of amyloidos-related diseases, such as for instance Alzheimer's disease, IgA nephropathy and type II diabetes.

The invention shall not be limited to the shown embodiments and examples.

### REFERENCES

1) Zerovnik A. Eur J Biochem 2002; 269:3362-3371
2) Merlini G and Belotti V. NEJM 2003; 349:583-596
3) Westermark P, Benson M D, Buxbaum J N, et al. Amyloid 2002; 9:197-200
4) McLaurin J, Yang D, Yip C M, et al. J Struct Biol 2000; 130:259-270
5) Schmidt M F G. Biochim. Biophys. Acta 1989; 988:411-426
6) Mclhinney R A J, Cdadwixk J K, and Pelly S J. Biochim. 1987; 244:109-115
7) Bhattacharya A A. J Mol Biol 2000; 303:721-732
8) Hermansen L F, Bergman T, Jornvall H, et al. Eur J Biochim 1995; 227:772-779
9) Miroy G J, Lai Z, Lashuel H A, et al. Proc Natl Acad Sci USA 1996; 93:15051-15056
10) Wilson D M and Binder L I. Am J Pathol 1997; 150:2181-2195)
11) Ma Z and Westermark G T. Medical Dissertation No. 655, Linkoping, Sweden 2001

1-36. (canceled)

37. A method for the treatment of amyloidos-related diseases, comprising administering to a human or an animal suffering from an amyloidos-related disease a fatty acid composition comprising (all-Z omega-3)-4,7,10,13,16,19-docosahexaenoic acid (DHA).

38. A method according to claim 37, wherein the fatty acid composition further comprises (all-Z omega-3)-5,8,11,14,17-eicosapentaenoic acid (EPA).

39. A method according to claim 38, wherein the weight ratio of EPA:DHA in the fatty acid composition is 1:X, where X is equal to or greater than 1.

40. A method according to claim 38, wherein the weight ratio of EPA:DHA in the fatty acid composition is from 1:1 to 1:8.

41. A method according to claim 37, wherein the fatty acids in the composition are presented in at least one of an esterified form, an ethyl ester form, a salt form, and a free acid form, or combination thereof.

42. A method according to claim 37, wherein at least DHA is obtained from at least one of a vegetable, a microbial, and an animal origin or a combination thereof.

43. A method according to claim 38, wherein at least one of DHA and EPA is obtained from at least one of a vegetable, a microbial, and an animal origin or a combination thereof.

44. A method according to claim 38, comprising EPA and/or DHA is obtained from a marine oil.

45. A method according to claim 37, wherein the fatty acid composition is obtained from a marine oil.

46. A method according to claim 38, wherein the fatty acid composition comprises EPA and DHA in a triglyceride form.

47. A method according to claim 37, wherein the fatty acid composition is administered orally to a human or an animal.

48. A method according to claim 37, wherein the fatty acid composition is administered in an amount providing a daily dosage of from 1 g to 15 g of said fatty acid composition.

49. A method according to claim 37, wherein the amyloidos-related disease is Alzheimer's disease.

50. A method according to claim 37, wherein the amyloidos-related disease is IgA nephropathy.

51. A method according to claim 37, wherein the amyloidos-related disease is type II diabetes mellitus.

52. A method according to claim 37, wherein the amyloidos-related disease is chosen from Parkinson's disease, kidney amyloidoses secondary to chronic inflammatory disease, or Creutzfeld-Jacob disease.

53. A method according to claim 37, wherein the administration to a human will prevent the formation of aggregates of protein fibrils or plaque and/or reduce deposed fibrils.

54. A method for treatment of Alzheimer's disease, comprising administering to a human or an animal suffering from Alzheimer's disease a fatty acid composition comprising (all-Z omega-3)-5,8,11,14,17-eicosapentaenoic acid (EPA) and (all-Z omega-3)-4,7,10,13,16,19-docosahexaenoic acid (DHA).

55. A method according to claim 54, wherein the EPA and DHA are present in the composition in an EPA:DHA ratio from 1:2 to 2:1.55.

56. A method according to claim 54, wherein the weight ratio of EPA:DHA in the fatty acid composition is 1:X, where X is equal to or greater than 1.

57. A method according to claim 56, wherein the weight ratio of EPA:DHA in the fatty acid composition is from 1:1 to 1:8.

58. A method according to claim 54, wherein the fatty acids in the composition are present in at least one of an esterified form, an ethyl ester form, a salt form, and a free acid form, or combinations thereof.

59. A method according to claim 54, wherein at least one of DHA and EPA is obtained from at least one of a vegetable, a microbial, and an animal origins or a combinations thereof.

60. A method according to claim 54, comprising EPA and/or DHA is obtained from a marine oil.

61. A method according to claim 54, wherein the fatty acid composition is obtained from a marine oil.

62. A method according to claim 54, wherein the fatty acid composition comprises EPA and DHA in a triglyceride form.

63. A method according to claim 54, wherein the fatty acid composition is administered orally to a human or an animal.

64. A method according to claim 54, wherein the fatty acid composition is administered in an amount providing a daily dosage of from 1 g to 15 g of said fatty acid composition.

65. A method according to claim 54, wherein the administration to a human will prevent misfolding of protein fibrils or plaque and/or reduce deposed fibrils or plaque.

66. A method for treatment of amyloidoses, comprising administering to a human or an animal suffering from amyloidoses a fatty acid composition comprising (all-Z omega-3)-4,7,10,13,16,19-docosahexaenoic acid (DHA).

67. A method according to claim 66, wherein the fatty acid composition further comprises (all-Z omega-3)-5,8,11,14,17-eicosapentaenoic acid (EPA).

68. A method according to claim 66, wherein the weight ratio of EPA:DHA in the fatty acid composition is 1:X, where X is equal to or greater than 1.

69. A method according to claim 38, wherein the weight ratio of EPA:DHA in the fatty acid composition is from 1:1 to 1:6.

70. A method according to claim 44, wherein the marine oil is a fish oil.

71. A method according to claim 48, wherein said fatty acid composition is administered in an amount providing a daily dosage of from 2 g to 6 g of said fatty acid composition.

72. A method according to claim 57, wherein the weight ratio of EPA:DHA in the fatty acid composition is from 1:1 to 1:6.

73. A method according to claim 64, wherein the fatty acid composition is administered in an amount providing a daily dosage of from 2 g to 6 g of said fatty acid composition.
